(19) United States
(12) Patent Application Publication
(10) Pub. No.: US 2014/0150186 A1
(43) Pub. Date: Jun. 5, 2014

(54) USE OF AN AGENT FOR KERATIN FIBERS, CONTAINING AT LEAST ONE NONIONIC STARCH MODIFIED BY MEANS OF PROPYLENE OXIDE AND AT LEAST ONE ADDITIONAL FILM-FORMING AND/OR STRENGTHENING POLYMER, FOR IMPROVING THE COLOR PRESERVATION OXIDATIVE HAIR COLORATIONS

(71) Applicant: Henkel AG & Co. KGaA, Duesseldorf (DE)

(72) Inventors: Diane Metten, Hamburg (DE); Rene Scheffler, Ellerau (DE)

(73) Assignee: Henkel AG & Co. KGaA, Duesseldorf (DE)

(21) Appl. No.: 14/174,908

(22) Filed: Feb. 7, 2014

Related U.S. Application Data
(63) Continuation of application No. PCT/EP2012/063942, filed on Jul. 17, 2012.

(30) Foreign Application Priority Data
Aug. 17, 2011 (DE) 10 2011 081 108.7

(51) Int. Cl. A61K 8/73; A61Q 5/00; A61K 8/84; A61Q 5/10

(52) U.S. Cl. CPC A61K 8/732; A61Q 5/10; A61Q 5/004; A61K 8/84
USPC 8/406

(57) ABSTRACT

Agents include in a cosmetically acceptable carrier (a) at least one non-ionic, propylene oxide-modified starch and (b) at least one film-forming and/or fixing polymer. Such agents improve the color retention of oxidatively dyed keratin-including fibers.

### FIELD OF THE INVENTION

The present invention generally relates to the use of a hair treatment agent including a combination of at least one non-ionic, propylene oxide-modified starch with at least one film-forming and/or fixing anionic polymer to improve the color retention of oxidative hair colorings, and to corresponding methods for treating keratin-including fibers.

### BACKGROUND OF THE INVENTION

Keratin-including fibers are understood in principle to be all animal hair, for example wool, horsehair, angora hair, fur, feathers and products or textiles manufactured therefrom. The keratinic fibers are however preferably human hair.

The coloring of keratinic fibers, for example human keratin fibers, generally takes place using coloring compositions including in addition to further constituents oxidation dye precursors, in particular oxidation bases (“developers”). Following the addition of an oxidizing agent as part of an oxidative condensation, these colorless or weakly colored substances react to form dye molecules. In order to shade the colors thus obtained, a second group of oxidation dye precursors known as “couplers” is generally added to the developers. A large number of different tints can be achieved by combining developer and coupler components.

In addition to or as an alternative to the aforementioned oxidation dyes, keratin fibers are colored by means of substantive dyes. These substantive dyes are colored molecules that attach to the surface of the keratin fibers.

The coloring of keratinic fibers obtained by means of oxidation dyes or substantive dyes fades under external influences such as light, but in particular through repeated hair washing.

To improve the color retention of oxidatively dyed keratin fibers, the use of zinc salts for example is proposed in European patents EP 1 915 981 B1 and EP 1 923 042 B1. According to the teaching of these documents, the zinc salts are applied to the keratinic fibers on completion of the coloring process.

However, the color retention values achieved according to the teaching of these documents are not satisfactory in every case.

In summary, there is therefore still a need for methods to improve the color retention of dyed keratinic fibers. Against this background it has been found that an improved color retention can be achieved by treating keratinic fibers with a combination of special polymers.

The present invention therefore firstly provides the use of an agent including, in a cosmetically acceptable carrier,

(a) at least one non-ionic, propylene oxide-modified starch and
(b) at least one film-forming and/or fixing anionic polymer
to improve the color retention of oxidatively dyed keratin-including fibers, in particular oxidatively dyed human hair.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

Use of an agent including, in a cosmetically acceptable carrier, (a) at least one non-ionic, propylene oxide-modified starch, and (b) at least one film-forming and/or fixing polymer to improve the color retention of oxidatively dyed keratin-including fibers, in particular oxidatively dyed human hair.

A method for treating keratin-including fibers, in particular human hair, comprising the following steps: i) performing an oxidative dyeing of the keratin-including fibers, ii) applying an agent including, in a cosmetically acceptable carrier, (a) at least one non-ionic, propylene oxide-modified starch, and (b) at least one film-forming and/or fixing polymer, to the oxidatively dyed keratin-including fibers.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Improving the color retention comprises according to the invention retaining the color on exposure to external influences. Color retention thus comprises, for example, phenomena such as wash fastness, light fastness, rubbing fastness or perspiration fastness of the oxidative hair coloring. The use according to the invention exhibits particular advantages in respect of color retention after washing the keratin fibers. The use of compositions according to the invention to improve the wash fastness of oxidatively dyed keratin-including fibers, in particular of oxidatively dyed human hair, is therefore a preferred embodiment of the present application.

Film-forming polymers are understood to be polymers which leave behind a continuous film on the skin, hair or nails when they dry. Such film formers can be used in a wide range of cosmetic products, such as for example face masks, make-up, hair fixing agents, hair sprays, hair gels, hair waxes, hair masks, shampoos or nail varnishes. Such polymers are preferred in particular which have an adequate solubility in water or water/alcohol mixtures in order for them to be present in the agent according to the invention in completely dissolved form. The film-forming polymers can be of synthetic or natural origin.

Film-forming polymers are also understood according to the invention to be polymers which when used in a 0.01 to 20 wt. % aqueous, alcoholic or aqueous-alcoholic solution are capable of depositing a transparent polymer film on the hair.

Fixing polymers contribute to the hold and/or to establishing volume and fullness in the hairstyle as a whole. These polymers are simultaneously also film-forming polymers and are therefore generally typical substances for hair treatment agents used for shaping, such as hair fixing agents, hair foams, hair waxes, hair sprays. Film formation may be entirely localized here and may bond only a few fibers together.

The three-point bending test or curl retention test is frequently used as a test method for the fixing effect of a polymer.

In the above formulae and all subsequent formulae, a chemical bond marked with the symbol * denotes a free valence of the corresponding structural fragment.

The properties of the agent according to the invention prove to be particularly advantageous if it is presented as an aerosol spray, aerosol foam, pump spray or pump foam. This preferred presentation form is described in detail further on.

Starch is a reserve carbohydrate which is stored by many plants in the form of starch granules of conventionally 1 to 200 μm in size in various parts of the plant, for example in tubers or roots, cereal seeds, fruits and in the pith. A non-ionic, propylene oxide-modified starch that can be used according to the invention can be derived from starch from potatoes, maize, rice, peas, acorns, chestnuts, barley, wheat, bananas, sago, millet, sorghum, oats, barley, rye, beans, yams, arrowroot or cassava. Particularly pronounced effects according to the invention are achieved through non-ionic, propylene oxide-modified tapioca starch or non-ionic, propylene oxide-modified potato starch or through mixtures of both of the aforementioned starches. The agent according to the invention particularly preferably includes at least one non-ionic, propylene oxide-modified potato starch.

Starch belongs to the homoglycan family and is a polycondensation product of D-glucose. Starch consists of three structurally different polymers of d-glucopyranose, namely amylose, amylopectin and an intermediate fraction. Relatively tall plants include 0 to 45 wt. % of amylose relative to dry matter.

In structural terms the intermediate fraction, which is also known as abnormal amylopectin, is between amylose and amylopectin. The amounts defined for amylopectin in the context of this application include the intermediate fraction.

It is preferable according to the invention for the non-ionic, propylene oxide-modified starch to have an amylose content of less than 25 wt. %, in particular less than 20 wt. %, relative in each case to the weight of the modified starch. It has been found that a starch including 17 to 22 wt. % of amylose and 78 to 83 wt. % of amylopectin is particularly suitable for achieving the effect according to the invention.

Amylose consists of d-glucose molecules linked by predominantly linear α-1,4-glycosidic bonds, Mᵣ 50,000-150,000. The resulting chains form double helixes in the starch.

In addition to the α-1,4 bonds described for amylose, amylopectin also includes from 4 to 6% of α-1,6 bonds as branch points. The average distance between the branch points is approximately 12 to 17 glucose units. The molar mass of 10⁷ to 7·10⁸ corresponds to approximately 10⁵ glucose units, making amylopectin one of the largest biopolymers. Said branches are distributed over the molecule in such a way that a cluster structure with relatively short side chains develops. Two such side chains form a double helix. The large number of branch points makes amylopectin relatively soluble in water.

A non-ionic, propylene oxide-modified starch is understood according to the invention to be a reaction product of a starch and propylene oxide. Such a reaction product comprises at least one structural unit of formula (PS),

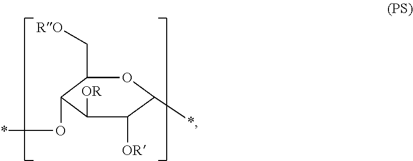

in which at least one residue R, R′ or R″ denotes a group of formula

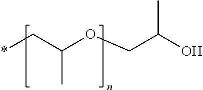

where n≧0
and a maximum of 2 of the residues R, R′, W′ denote a hydrogen atom. The non-ionic, propylene oxide-modified starches are prepared by reacting a native starch with propylene oxide, for example. Before being modified with propylene oxide, the starch can be exposed to various physical or chemical processes, such as for example heat treatment, shearing, or thermal, acid-hydrolytic, oxidative or enzymatic cleavage, etc.

It is preferable according to the invention for the non-ionic, propylene oxide-modified starch not to be present in the agent according to the invention in the form of individual starch granules. To this end the starch granules are broken down by heat or shearing, for example, and the corresponding polysaccharide molecules are released from the bond. The released polysaccharide molecules are modified with propylene oxide after or before being released.

In a preferred embodiment the non-ionic, propylene oxide-modified starch is gelatinized. If an aqueous suspension of starch is heated or compressed, a tangential swelling of the bodies with loss of double refraction, change to the X-ray structure and a sharp rise in the viscosity of the solution is observed at a critical temperature or pressure. This phenomenon is known as gelatinization.

The non-ionic, propylene oxide-modified starches according to the invention are present in the agent according to the invention in a molecular weight distribution. Non-ionic, propylene oxide-modified starches that are preferably used according to the invention have an average molecular weight of 50 to 2500 kDa (weight average). The molecular weight distribution was determined experimentally by gel filtration chromatography against dextran. Said weight average is an average molecular weight that takes account not only of the number of molecules but also of the total weight of molecules of varying molecular weight. The first step in the statistical calculation of the weight average is to define the “weight fraction”

wᵢ=(NᵢMᵢ)/[Σ(NᵢMᵢ)]

This indicates the percentage by weight in the sample of macromolecules that consist of i segments (e.g. monomer units) of mass Mᵢ and occur Nᵢ times in the sample. Thus for the weight average of the molecular weight M_{w}=ΣwᵢMᵢ

M_{w}=[Σ(NᵢM²ᵢ)]/[Σ(NᵢMᵢ)].

Agents that are more preferably used according to the invention include such non-ionic, propylene oxide-modified starches with an average molecular weight (weight average) of 100 to 2000 kDa, in particular 500 to 1800 kDa, particularly preferably 700 to 1000 kDa.

In order to adjust the molecular weight, the starch undergoes a mechanical and/or chemical treatment before or after being modified with propylene oxide. To increase the molecular weight, said starch can be crosslinked. The non-ionic, propylene oxide-modified starch is crosslinked if the linear or branched polysaccharide macromolecules of the starch are covalently bonded by a crosslinking agent to form a three-dimensional, insoluble and only swellable polymeric network. Native starch is generally considered to be uncrosslinked and, if crosslinking is desired, requires artificial crosslinking by means of synthetic chemistry. Such an artificial crosslinking can be performed with crosslinking agents. (Non-ionic, propylene oxide-modified) starches that do not exhibit such a crosslinking are uncrosslinked.

Crosslinking can be carried out using the crosslinking agent epichlorohydrin, for example. To this end a 42 wt. % mixture of non-ionic, propylene oxide-modified starch in water is prepared, into which the desired amount of epichlorohydrin is stirred at room temperature and, after a stirring time of 1 to 5 hours with viscosity control, the crosslinked starch is isolated by customary methods on reaching the target viscosity.

It is however more preferable according to the invention for the agents used according to the invention to include at least one uncrosslinked, non-ionic, propylene oxide-modified starch.

In order to achieve a lower molecular weight of 100 to 400 kDa, said starches preferably undergo a mechanical cleavage, an enzymatic cleavage (in particular with alpha-amylase, beta-amylase, glucoamylase or debranching enzymes), an acid-hydrolytic cleavage (in particular with hydrochloric acid, sulfuric acid or phosphoric acid), a thermal cleavage or a reaction with oxidizing agents (such as periodate, hypochlorite, chromic acid, permanganate, nitrogen dioxide, hydrogen peroxide or organic percarboxylic acid, preferably with hydrogen peroxide). Kneaders, extruders, stator/rotor machines and/or agitators are suitable for the mechanical cleavage of starch.

Oxidative cleavage using hydrogen peroxide is preferred according to the invention. To this end, for example, the non-ionic, propylene oxide-modified starch is introduced into water, heated to 50 to 70° C., hydrogen peroxide is added and the mixture is stirred at 70 to 85° C. for 2 to 5 hours.

The propylene oxide content of the starch affects the precision adjustment of the styling hold along with the styling flexibility and the stability of the cosmetic agents. It has been found that the parameters can be further optimized if the non-ionic, propylene oxide-modified starch has a propylene oxide content, relative to the weight of the modified starch, of 1 to 20 wt. %, more preferably a propylene oxide content of 4 to 12 wt. %, particularly preferably a propylene oxide content of 9.5 to 10.5 wt. % or 4.0 to 6.0 wt. %. The propylene oxide content can be determined for example after performing a Hodges cleavage by the method according to DIN EN 13268.

It has further been found that cosmetic agents in which the non-ionic, propylene oxide-modified starch in a 43 wt. % aqueous solution has a preferred viscosity in the range from 150 to 1,500,000 mPa·s (Brookfield viscometer, spindle 7 at 20° C. and 20 rpm) are outstandingly suitable for use according to the invention. Particularly suitable propylene oxide-modified starches have viscosities from 10,000 to 200,000 mPa·s, more preferably from 25,000 to 180,000 mPa·s (measured in each case under the aforementioned conditions).

A non-ionic, propylene oxide-modified starch that is more preferred according to the invention is uncrosslinked, has an average molecular weight (weight average) of 100 to 2000 kDa, in particular 500 to 1800 kDa, particularly preferably 700 to 1000 kDa, and has a propylene oxide content, relative to the weight of the modified starch, of 1 to 20 wt. %, more preferably a propylene oxide content of 4 to 12 wt. %, particularly preferably a propylene oxide content of 9.5 to 10.5 wt. % or 4.0 to 6.0 wt. %. The starch is preferably in turn tapioca starch or potato starch, in particular potato starch.

A non-ionic, propylene oxide-modified starch that is more preferably used according to the invention is uncrosslinked, has an average molecular weight (weight average) of 100 to 2000 kDa, in particular 500 to 1800 kDa, particularly preferably 700 to 1000 kDa, and has a propylene oxide content, relative to the weight of the modified starch, of 1 to 20 wt. %, more preferably a propylene oxide content of 4 to 12 wt. %, particularly preferably a propylene oxide content of 9.5 to 10.5 wt. % or 4.0 to 6.0 wt. %. The starch is preferably in turn tapioca starch or potato starch, in particular potato starch.

A non-ionic, propylene oxide-modified potato starch that is particularly preferably used according to the invention is uncrosslinked, has an average molecular weight (weight average) of 100 to 2000 kDa, in particular 500 to 1800 kDa, particularly preferably 700 to 1000 kDa, and has a propylene oxide content, relative to the weight of the modified potato starch, of 4 to 12 wt. %, particularly preferably a propylene oxide content of 9.5 to 10.5 wt. % or 4.0 to 6.0 wt. %.

It is preferable according to the invention for the cosmetic agent that is used according to the invention to include the non-ionic, propylene oxide-modified starch in an amount from 1.0 wt. % to 20 wt. %, more preferably from 4.0 wt. % to 15 wt. %, particularly preferably from 8.0 to 12 wt. %, relative in each case to the weight of the agent.

The agent used according to the invention must additionally include at least one film-forming and/or fixing polymer.

Preferred film-forming and/or fixing polymers are copolymers of maleic anhydride and methyl vinyl ether.

More preferred film-forming and/or fixing polymers are anionic. An anionic polymer is understood according to the invention to be a polymer which in a protic solvent under standard conditions bears structural units having anionic groups needing to be offset by counterions to maintain electrical neutrality and has no structural units having permanently cationic groups. Anionic groups include carboxyl and sulfonic acid groups.

Film-forming anionic and/or fixing anionic polymers have proved to be particularly effective that comprise at least one structural unit of formula (I) and at least one structural unit of formula (II)

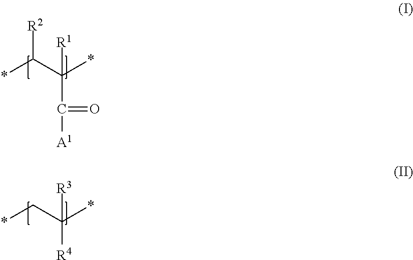

in which
R¹ and R² independently of one another denote a hydrogen atom or a methyl group, with the proviso that R¹ and R² do not simultaneously denote a methyl group,
R³ denotes a hydrogen atom or a methyl group,
R⁴ denotes a carbamoyl group, a linear or branched (C₄ to C₁₂) alkyl aminocarbonyl group, a linear or branched (C₄ to C₁₂) alkyl aminoethyl aminocarbonyl group, a linear or branched (C₄ to C₁₂) alkyl aminopropyl aminocarbonyl group, a linear or branched (C₄ to C₁₂) alkyloxycarbonyl group, a linear or branched (C₄ to C₁₂) alkyl aminoethyl oxycarbonyl group, a linear or branched (C₄ to C₁₂) alkyl aminopropyl oxycarbonyl group, a linear or branched (C₂ to C₁₂) acyloxy group,
A¹ denotes a hydroxyl group or an organic residue having at least one sulfonic acid group, which binds to the structural fragment via an oxygen atom or an NH group.

The film-forming and/or fixing, preferably anionic film-forming and/or anionic fixing polymers (b) are preferably included in the agent used according to the invention in an amount from 0.5 wt. % to 10 wt. %, more preferably from 1.0 wt. % to 9.0 wt. %, particularly preferably from 3.0 wt. % to 8.0 wt. %, relative in each case to the weight of the agent used according to the invention.

It is preferable according to the invention for the film-forming anionic and/or fixing anionic polymer (b) to include at least one structural unit of formula (I), which is selected from at least one structural unit of formulae (I-1) to (I-5)

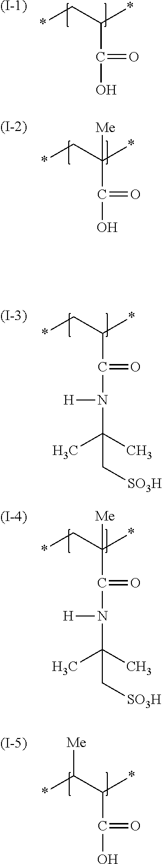

It is more preferable in turn for the polymer (b) additionally to include, in addition to the above structural units of formulae (I) and (II), at least one structural unit of formula (III)

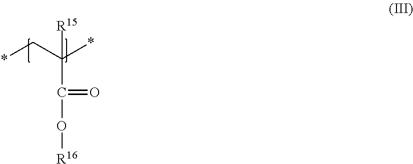

in which
R¹⁵ denotes a hydrogen atom or a methyl group
R¹⁶ denotes a (C₁ to C₄) alkyl group (in particular a methyl group or an ethyl group).

Copolymers of methacrylic acid and ethyl acrylate and tert-butyl acrylate for example are suitable as being preferred.

It is more preferable according to the invention for the film-forming anionic and/or fixing anionic polymer (b) to include at least one structural unit of formula (II), which is selected from at least one structural unit of formulae (II-1) to (II-15).

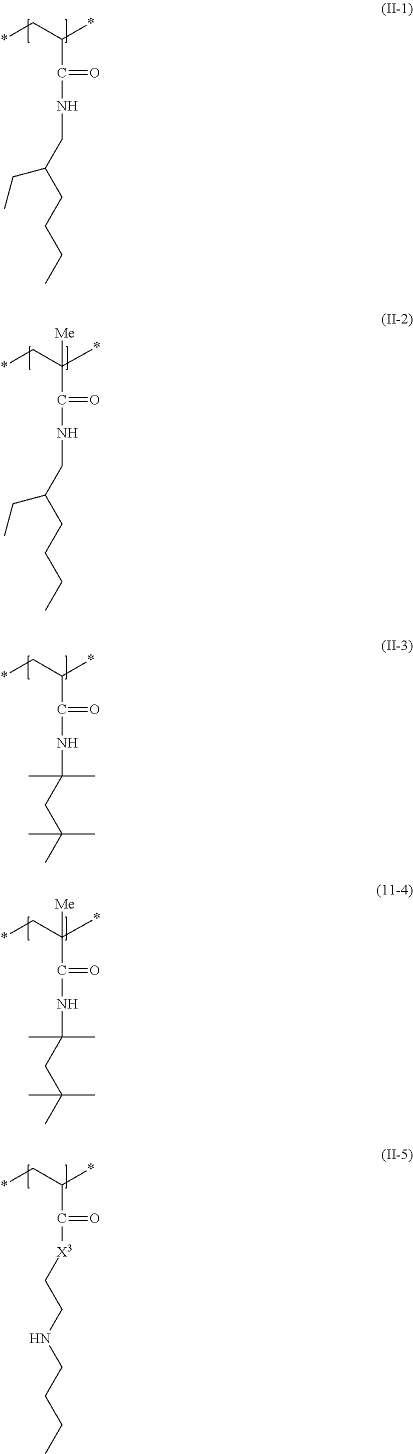

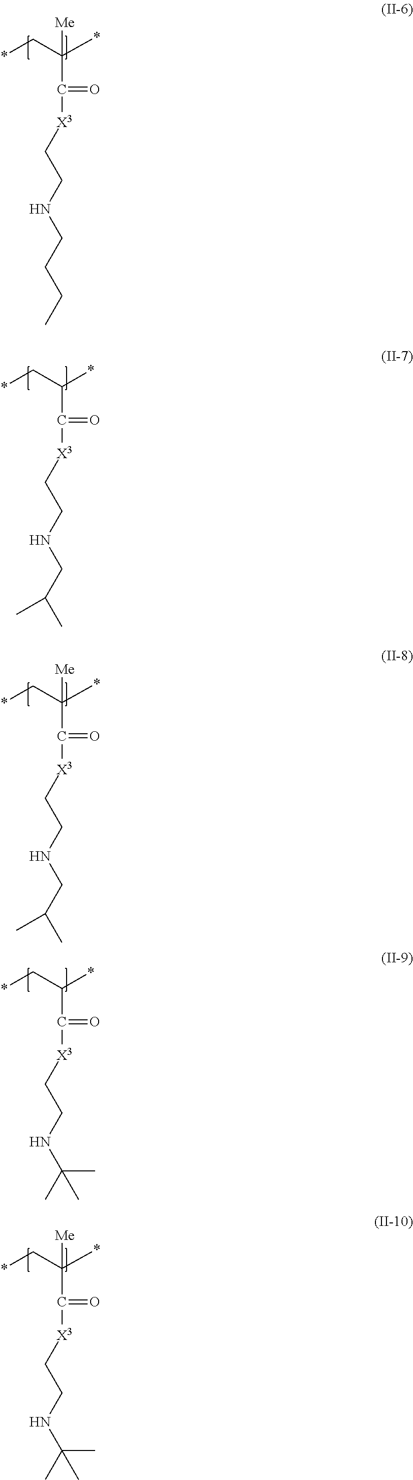

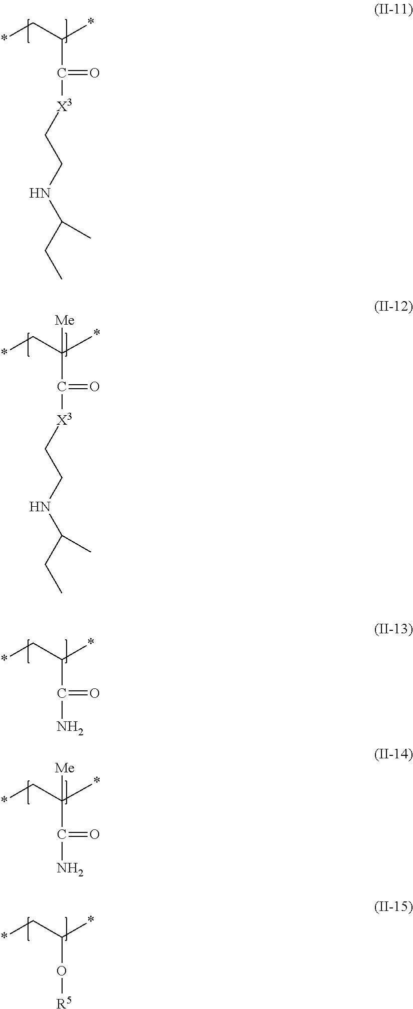

in which
X³ denotes an oxygen atom or an NH group,
R⁵ denotes a (C₂ to C₁₂) acyl group (in particular acetyl or neodecanoyl).

It is preferable according to the invention for X³ according to formulae (II-5) to (II-12) to denote an oxygen atom.

In the context of a first preferred embodiment of the invention the agent used according to the invention includes at least one film-forming anionic and/or fixing anionic polymer (b) including at least one structural unit of formula (I-1), at least one structural unit of formula (II-3) and at least one structural unit of formula (II-16) (selected in particular from the group formed from the above formulae (II-5) to (II-12) with the proviso that X³ denotes an oxygen atom),

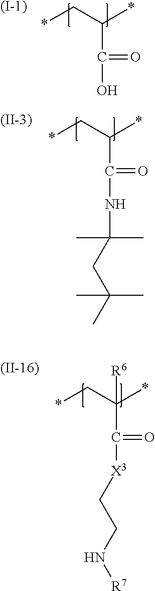

in which X³ denotes an oxygen atom or an NH group,
R⁶ denotes a hydrogen atom or a methyl group and
R⁷ denotes an alkyl group having 4 carbon atoms (in particular n-butyl, sec-butyl, isobutyl or tert-butyl).

It is more preferable in turn for the polymer (b) additionally to include, in addition to the above structural units of formulae (I-1), (II-3) and (II-16), at least one structural unit of formula (III)

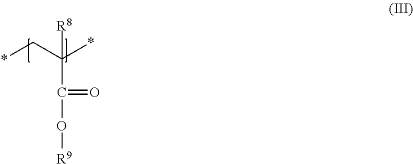

in which
R⁸ denotes a hydrogen atom or a methyl group
R⁹ denotes a (C₁ to C₄) alkyl group (in particular a methyl group or an ethyl group).

Preferred polymers (b) of this type are selected from the group formed from:
copolymers of acrylic acid, (C₁ to C₄) alkyl acrylates, C₄ alkyl aminoethyl methacrylate and C₈ alkyl acrylamide.

One example of a polymer (b) that can more preferably be used in the context of this embodiment is the polymer obtainable under the commercial name Amphomer® 028-4910 from National Starch with the INCI name Octylacrylamide/Acrylates/Butylaminoethylmethacrylate Copolymer.

In particular, agents that are particularly preferred in the context of this embodiment are thus those which in a cosmetically acceptable carrier include
(a) at least one uncrosslinked, non-ionic, propylene oxide-modified starch, in particular with an average molecular weight (weight average) of 50 to 2500 kDa,
   and
(b) at least one anionic film-forming and/or anionic fixing polymer (b) comprising at least one structural unit of formula (I-1), at least one structural unit of formula (II-3) and at least one structural unit of formula (II-16),

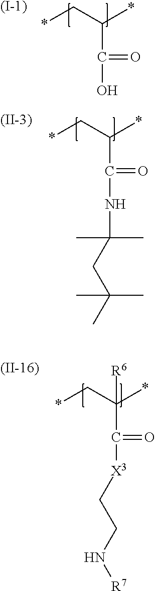

in which
X³ denotes an oxygen atom or an NH group (in particular an oxygen atom),
R⁶ denotes a hydrogen atom or a methyl group (in particular a methyl group) and
R⁷ denotes an alkyl group having 4 carbon atoms (in particular n-butyl, sec-butyl, isobutyl or tert-butyl).

It is more preferable in turn for the polymer (b) additionally to include, in addition to the above structural units of formulae (I-1), (II-3) and (II-16), at least one structural unit of formula (III)

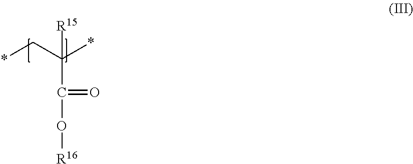

in which
R¹⁵ denotes a hydrogen atom or a methyl group
R¹⁶ denotes a (C₁ to C₄) alkyl group (in particular a methyl group or an ethyl group).

It is more preferable for all embodiments of this first embodiment if the structural unit of formula (I-1) is wholly or partially neutralized. At least one alkanol amine is preferably used for neutralization. The alkanol amines for use as the alkalizing agent according to the invention are preferably selected from primary amines having a C₂-C₆ alkyl parent substance bearing at least one hydroxyl group. More preferred alkanol amines are selected from the group formed from 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, 2-amino-2-methylpropane-1,3-diol. Particularly preferred alkanol amines according to the invention are selected from the group comprising 2-aminoethan-1-ol, 2-amino-2-methylpropan-1-ol and 2-amino-2-methylpropane-1,3-diol.

In the context of a second embodiment agents are preferred according to the invention which include as the anionic film-forming and/or anionic fixing polymer (b) at least one polymer including at least one structural unit of formula (I-3) and at least one structural unit of formula (I-13)

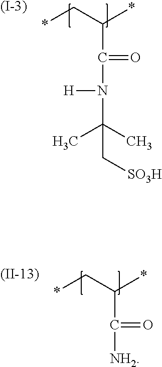

Preferred polymers (b) of this type are selected from at least one polymer of the group formed from
copolymers of 2-acrylamido-2-methylpropane sulfonic acid and acrylamide,
copolymers of 2-acrylamido-2-methylpropane sulfonic acid, acrylamide and acrylic acid.

Polymers of this type are sold for example in an invert isohexadecane emulsion by Seppic under the trade name Sepigel® 305 (INCI name: Polyacrylamide, C13-14 Isoparaffin, Laureth-7) or Simulgel® 600 (INCI name: Acrylamide/Acryloyldimethyltaurate Copolymer, Isohexadecane, Polysorbate-80).

An agent that is more preferably used according to the invention is characterized in that it includes as the polymer (b) a copolymer (b1).

These copolymers (b1) can be described by the general formula

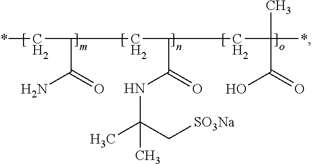

in which the indices m, n and o vary according to the molar mass of the polymer and are not intended to suggest that the copolymers are block copolymers. The structural units can rather be randomly distributed in the molecule.

Agents that are more preferably used according to the invention are characterized in that the copolymer (b1) has a molar mass of 50 to 500 kDa, preferably 100 to 450 kDa, more preferably 150 to 400 kDa and in particular 200 to 300 kDa.

Copolymers of acrylamide with methacrylic acid and acryloyldimethyltaurate are available for example under the trade name Acudyne® SCP (Rohm & Haas).

Therefore, in the context of this embodiment, agents that are particularly preferred are furthermore in particular those which in a cosmetically acceptable carrier include
(a) at least one uncrosslinked, non-ionic, propylene oxide-modified starch, in particular with an average molecular weight (weight average) of 50 to 2500 kDa,
   and
(b) at least one anionic film-forming and/or anionic fixing polymer (b) comprising at least one structural unit of formula (I-5) and at least one structural unit of formula

(I-13),

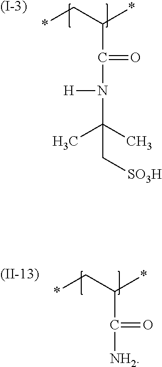

In the context of a third embodiment agents are preferred according to the invention which include as the anionic film-forming and/or anionic fixing polymer (b) at least one polymer including at least one structural unit of formula (I-3) and at least one structural unit of formula (I-13)

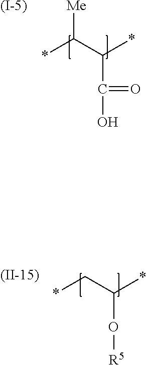

in which R⁵ denotes a (C₂ to C₁₂) acyl group (in particular acetyl or neodecanoyl). Particularly preferred polymers (b) of this type are selected from at least one polymer of the group formed from
copolymers of vinyl acetate and crotonic acid,
copolymers of vinyl propionate and crotonic acid,
copolymers of vinyl neodecanoate, vinyl acetate and crotonic acid.

Such copolymers are provided for example by Clariant under the trade name Aristoflex A 60 (INCI name: VA/Crotonates Copolymer) in an isopropanol/water blend (60 wt. % active substance), by BASF under the trade name Luviset CA 66 (vinyl acetate/crotonic acid copolymer 90:10, INCI name VA/Crotonates Copolymer), by National Starch under the trade name Resyn 28-2942 and Resyn 28-2930 (INCI name: VA/Crotonates/Vinyl Neodecanoate Copolymer).

Therefore, in the context of this embodiment, agents that are particularly preferred are furthermore in particular those which in a cosmetically acceptable carrier include
(a) at least one uncrosslinked, non-ionic, propylene oxide-modified starch, in particular with an average molecular weight (weight average) of 50 to 2500 kDa,
   and
(b) at least one anionic film-forming and/or anionic fixing polymer (b) comprising at least one structural unit of formula (I-5) and at least one structural unit of formula (I-15),

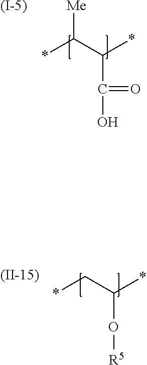

in which R⁵ denotes a (C₂ to C₁₂) acyl group (in particular acetyl or neodecanoyl).

In the context of this embodiment the aforementioned preferred embodiments of the amphiphilic, cationic polymer (a) are preferred (see above). All aforementioned preferred amounts in respect of the polymer components (a) and (b) for the use of the agent according to the invention are also preferred for these embodiments with necessary alterations.

It is more preferable for all embodiments of this third embodiment if the structural unit of formula (I-5) is wholly or partially neutralized. At least one alkanol amine is preferably used for neutralization. The alkanol amines for use as the alkalizing agent according to the invention are preferably selected from primary amines having a C₂-C₆ alkyl parent substance bearing at least one hydroxyl group. Particularly preferred alkanol amines are selected from the group formed from 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, 2-amino-2-methylpropane-1,3-diol. Particularly preferred alkanol amines according to the invention are selected from the group comprising 2-aminoethan-1-ol, 2-amino-2-methylpropan-1-ol and 2-amino-2-methylpropane-1,3-diol.

To intensify the effect according to the invention the agents according to the invention preferably additionally include at least one surfactant, with non-ionic, anionic, cationic and ampholytic surfactants being suitable in principle. The group of ampholytic or amphoteric surfactants comprises zwitterionic surfactants and ampholytes. According to the invention the surfactants can already have an emulsifying action.

The additional surfactants are preferably included in the agent used according to the invention in an amount from 0.01 wt. % to 5 wt. %, more preferably from 0.05 wt. % to 0.5 wt. %, relative in each case to the weight of the agent.

It has proved particularly preferable for the agents used according to the invention additionally to include at least one non-ionic surfactant.

Non-ionic surfactants include as a hydrophilic group a polyol group, a polyalkylene glycol ether group or a combination of a polyol and polyglycol ether group, for example. Such compounds are for example
addition products of 2 to 100 mol of ethylene oxide and/or 1 to 5 mol of propylene oxide with linear and branched fatty alcohols having 8 to 30 C atoms, with fatty acids having 8 to 30 C atoms and with alkylphenols having 8 to 15 C atoms in the alkyl group,
addition products of 2 to 50 mol of ethylene oxide and/or 1 to 5 mol of propylene oxide with linear and branched fatty alcohols having 8 to 30 C atoms, with fatty acids having 8 to 30 C atoms and with alkylphenols having 8 to 15 C atoms in the alkyl group, end-capped with a methyl or C₂ to C₆ alkyl residue, such as for example the types available under the commercial names Dehydol® LS, Dehydol® LT (Cognis),
C₁₂-C₃₀ fatty acid monoesters and diesters of addition products of 1 to 30 mol of ethylene oxide with glycerol,
addition products of 5 to 60 mol of ethylene oxide with castor oil and hydrogenated castor oil,
polyol fatty acid esters, such as for example the commercial product Hydagen® HSP (Cognis) or Sovermol types (Cognis),
alkoxylated triglycerides,
alkoxylated fatty acid alkyl esters of formula (E4-I)

R¹CO—(OCH₂CHR²)_{w}OR³ (E4-I)
in which R¹CO denotes a linear or branched, saturated and/or unsaturated acyl residue having 6 to 22 carbon atoms, R² denotes hydrogen or methyl, R³ denotes linear or branched alkyl residues having 1 to 4 carbon atoms and w denotes numbers from 1 to 20,
amine oxides,
hydroxy mixed ethers, such as are described for example in DE-OS 19738866,
sorbitan fatty acid esters and addition products of ethylene oxide with sorbitan fatty acid esters such as for example polysorbates,
sugar fatty acid esters and addition products of ethylene oxide with sugar fatty acid esters,
addition products of ethylene oxide with fatty acid alkanol amides and fatty amines,
sugar surfactants of the alkyl and alkenyl oligoglycoside type according to formula (E4-II),

R⁴O-[G]ₚ (E4-II)
in which R⁴ denotes an alkyl or alkenyl residue having 4 to 22 carbon atoms, G denotes a sugar residue having 5 or 6 carbon atoms and p denotes numbers from 1 to 10. They can be obtained by means of the relevant methods of preparative organic chemistry.

The alkylene oxide addition products with saturated linear fatty alcohols and fatty acids each including 2 to 100 mol of ethylene oxide per mol of fatty alcohol or fatty acid have proved themselves to be particularly preferred non-ionic surfactants. Preparations having outstanding properties are likewise obtained if they include C₁₂-C₃₀ fatty acid mono- and diesters of addition products of 1 to 30 mol of ethylene oxide with glycerol and/or addition products of 5 to 60 mol of ethylene oxide with castor oil and hydrogenated castor oil as non-ionic surfactants.

The agents according to the invention particularly preferably include at least one addition product of 15 to 100 mol of ethylene oxide, in particular of 15 to 50 mol of ethylene oxide, with a linear or branched (in particular linear) fatty alcohol having 8 to 22 carbon atoms as the surfactant. This is particularly preferably Ceteareth-15, Ceteareth-25 or Ceteareth-50, which are sold respectively as Eumulgin® CS 15 (COGNIS), Cremophor A25 (BASF SE) and Eumulgin® CS 50 (COGNIS).

All anionic surface-active substances which are suitable for use on the human body are suitable in principle as anionic surfactants. These are characterized by a water-solubilizing anionic group such as for example a carboxylate, sulfate, sulfonate or phosphate group and a lipophilic alkyl group having approximately 8 to 30 C atoms. The molecule can additionally include glycol or polyglycol ether groups, ester, ether and amide groups and hydroxyl groups. Examples of suitable anionic surfactants, each in the form of the sodium, potassium and ammonium salts as well as the mono-, di- and trialkanolammonium salts having 2 to 4 C atoms in the alkanol group, are
linear and branched fatty acids having 8 to 30 C atoms (soaps),
ether carboxylic acids of the formula R—O—(CH₂—CH₂O)ₓ—CH₂—COOH, in which R is a linear alkyl group having 8 to 30 C atoms and x=0 or 1 to 16,
acyl sarcosides having 8 to 24 C atoms in the acyl group,
acyl taurides having 8 to 24 C atoms in the acyl group,
acyl isethionates having 8 to 24 C atoms in the acyl group,
sulfosuccinic acid mono- and dialkyl esters having 8 to 24 C atoms in the alkyl group and sulfosuccinic acid monoalkyl polyoxyethyl esters having 8 to 24 C atoms in the alkyl group and 1 to 6 oxyethyl groups,
linear alkane sulfonates having 8 to 24 C atoms,
linear alpha-olefin sulfonates having 8 to 24 C atoms,
alpha-sulfo fatty acid methyl esters of fatty acids having 8 to 30 C atoms,
alkyl sulfates and alkyl polyglycol ether sulfates of the formula R—O(CH₂—CH₂O)ₓ—OSO₃H, in which R is a preferably linear alkyl group having 8 to 30 C atoms and x=0 or 1 to 12,
mixtures of surface-active hydroxyl sulfonates,
sulfated hydroxyalkyl polyethylene and/or hydroxyalkylene propylene glycol ethers,
sulfonates of unsaturated fatty acids having 8 to 24 C atoms and 1 to 6 double bonds,
esters of tartaric acid and citric acid with alcohols that are addition products of around 2 to 15 molecules of ethylene oxide and/or propylene oxide with fatty alcohols having 8 to 22 C atoms,
sulfated fatty acid alkylene glycol esters of formula (E1-II)

R⁷CO(AlkO)ₙSO₃M (E1-II)
in which R⁷CO denotes a linear or branched, aliphatic, saturated and/or unsaturated acyl residue having 6 to 22 C atoms, Alk denotes CH₂CH₂, CHCH₃CH₂ and/or CH₂CHCH₃, n denotes numbers from 0.5 to 5 and M denotes a cation such as are described in DE-OS 197 36 906,
amide ether carboxylic acids,
condensation products of C₈ to C₃₀ fatty alcohols with protein hydrolysates and/or amino acids and derivatives thereof, which are known to the person skilled in the art as protein fatty acid condensates, such as for example the Lamepon® types, Gluadin® types, Hostapon® KCG or Amisoft® types.

Preferred anionic surfactants are alkyl sulfates, alkyl polyglycol ether sulfates and ether carboxylic acids having 10 to 18 C atoms in the alkyl group and up to 12 glycol ether groups in the molecule, sulfosuccinic acid mono- and dialkyl esters having 8 to 18 C atoms in the alkyl group and sulfosuccinic acid monoalkyl polyoxyethyl esters having 8 to 18 C atoms in the alkyl group and 1 to 6 oxyethyl groups, monoglyceride sulfates, alkyl and alkenyl ether phosphates and protein fatty acid condensates.

Also suitable for use according to the invention are cationic surfactants of the quaternary ammonium compound, esterquat and amidoamine type. Preferred quaternary ammonium compounds are ammonium halides, in particular chlorides and bromides, such as alkyl trimethylammonium chlorides, dialkyl dimethylammonium chlorides and trialkyl methylammonium chlorides. The long alkyl chains of these surfactants preferably have 10 to 18 carbon atoms, such as for example in cetyl trimethylammonium chloride, stearyl trimethylammonium chloride, distearyl dimethylammonium chloride, lauryl dimethylammonium chloride, lauryl dimethyl benzylammonium chloride and tricetyl methylammonium chloride. Further preferred cationic surfactants are the imidazolium compounds known under the INCI names Quaternium-27 and Quaternium-83.

Surface-active compounds classed as zwitterionic surfactants are those bearing at least one quaternary ammonium group and at least one —COO⁽⁻⁾ or —SO₃⁽⁻⁾ group in the molecule. Particularly suitable zwitterionic surfactants are the betaines such as N-alkyl-N,N-dimethylammonium glycinates, for example cocoalkyl dimethylammonium glycinate, N-acyl aminopropyl-N,N-dimethylammonium glycinates, for example cocoacylaminopropyl dimethylammonium glycinate, and 2-alkyl-3-carboxymethyl-3-hydroxyethyl imidazolines each having 8 to 18 C atoms in the alkyl or acyl group, and cocoacylaminoethyl hydroxyethyl carboxymethyl glycinate. A preferred zwitterionic surfactant is the fatty acid amide derivative known under the INCI name Cocamidopropyl Betaine.

Ampholytes are understood to be surface-active compounds which in addition to a C₈-C₂₄ alkyl or acyl group include at least one free amino group and at least one —COOH or —SO₃H group in the molecule and are capable of forming internal salts. Examples of suitable ampholytes are N-alkyl glycines, N-alkyl propionic acids, N-alkyl aminobutyric acids, N-alkyl iminodipropionic acids, N-hydroxyethyl-N-alkyl amidopropyl glycines, N-alkyl taurines, N-alkyl sarcosines, 2-alkyl aminopropionic acids and alkyl aminoacetic acids, each having approximately 8 to 24 C atoms in the alkyl group. More preferred ampholytes are N-cocoalkyl aminopropionate, cocoacylaminoethyl aminopropionate and C₁₂-C₁₈ acyl sarcosine.

The agents used according to the invention include the ingredients and active ingredients in a cosmetically acceptable carrier.

Preferred cosmetically acceptable carriers are aqueous, alcoholic or aqueous-alcoholic media having preferably at least 10 wt. % water, relative to the total agent. The low alcohols having 1 to 4 carbon atoms that are conventionally used for cosmetic purposes, such as for example ethanol and isopropanol, can be included in particular as alcohols.

It is preferable according to the invention to use at least one (C₁ to C₄) monoalkyl alcohol in the agents according to the invention, in particular in an amount from 1 to 50 wt. %, in particular from 5 to 30 wt. %. This is in turn preferable in particular for the presentation as a pump foam or aerosol foam.

Organic solvents or a mixture of solvents with a boiling point below 400° C. can be included as additional co-solvents in an amount from 0.1 to 15 percent by weight, preferably from 1 to 10 percent by weight, relative to the total agent. Unbranched or branched hydrocarbons, such as pentane, hexane, isopentane, and cyclic hydrocarbons, such as cyclopentane and cyclohexane, are particularly suitable as additional co-solvents.

Other more preferred water-soluble solvents are glycerol, ethylene glycol and propylene glycol in an amount of up to 30 wt. % relative to the total agent.

In particular, the addition of glycerol and/or propylene glycol and/or polyethylene glycol and/or polypropylene glycol increases the flexibility of the polymer film formed on application of the agent according to the invention. If a flexible hold is desired, the agents according to the invention therefore preferably include 0.01 to 30 wt. % of glycerol and/or propylene glycol and/or polyethylene glycol and/or polypropylene glycol, relative to the total agent.

The agents preferably have a pH of 2 to 11. The pH range between 4.0 and 9.0 preferably between 4.5 and 7.5 and in particular between 5.0 and 6.0 is more preferred. Unless otherwise specified, within the meaning of this document the stated pH values relate to the pH at 25° C.

The agents used according to the invention can furthermore include the auxiliary substances and additives that are conventionally added to customary styling agents.

Suitable auxiliary substances and additives include in particular additional care substances.

A silicone oil and/or a silicone gum for example can be used as a care substance.

Suitable silicone oils or silicone gums according to the invention are in particular dialkyl and alkylaryl siloxanes, such as for example dimethyl polysiloxane and methyl phenyl polysiloxane, and the alkoxylated, quaternized or also anionic derivatives thereof. Cyclic and linear polydialkyl siloxanes, the alkoxylated and/or aminated derivatives thereof, dihydroxypolydimethyl siloxanes and polyphenyl alkyl siloxanes are preferred.

Silicone oils give rise to a wide variety of effects. Thus for example they influence simultaneously the dry and wet combability of hair, the feel of dry and wet hair and the shine. The term silicone oils is understood by the person skilled in the art to mean a plurality of structures of organosilicon compounds. They are firstly understood to be dimethiconols.

The following commercial products are cited as examples of such products: Botanisil NU-150M (Botanigenics), Dow Corning 1-1254 Fluid, Dow Corning 2-9023 Fluid, Dow Corning 2-9026 Fluid, Ultrapure Dimethiconol (Ultra Chemical), Unisil SF-R (Universal Preserve), X-21-5619 (Shin-Etsu Chemical Co.), Abil OSW 5 (Degussa Care Specialties), ACC DL-9430 Emulsion (Taylor Chemical Company), AEC Dimethiconol & Sodium Dodecylbenzenesulfonate (A & E Connock (Perfumery & Cosmetics) Ltd.), B C Dimethiconol Emulsion 95 (Basildon Chemical Company, Ltd.), Cosmetic Fluid 1401, Cosmetic Fluid 1403, Cosmetic Fluid 1501, Cosmetic Fluid 1401DC (all of the above Chemsil Silicones, Inc.), Dow Corning 1401 Fluid, Dow Corning 1403 Fluid, Dow Corning 1501 Fluid, Dow Corning 1784 HVF Emulsion, Dow Corning 9546 Silicone Elastomer Blend (all of the above Dow Corning Corporation), Dub Gel Sl 1400 (Stearinerie Dubois Fils), HVM 4852 Emulsion (Crompton Corporation), Jeesilc 6056 (Jeen International Corporation), Lubrasil, Lubrasil DS (both Guardian Laboratories), Nonychosine E, Nonychosine V (both Exsymol), SanSurf Petrolatum-25, Satin Finish (both Collaborative Laboratories, Inc.), Silatex-D30 (Cosmetic Ingredient Resources), Silsoft 148, Silsoft E-50, Silsoft E-623 (all of the above Crompton Corporation), SM555, SM2725, SM2765, SM2785 (all of the above GE Silicones), Taylor T-Sil CD-1, Taylor TME-4050E (all Taylor Chemical Company), TH V 148 (Crompton Corporation), Tixogel CYD-1429 (Sud-Chemie Performance Additives), Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, Wacker-Belsil DM 60081 VP (all of the above Wacker-Chemie GmbH).

Dimethicones form the second group of silicones that can be included according to the invention. These can be both linear and branched and also cyclic or cyclic and branched.

Dimethicone copolyols (S3) are a further group of silicones that are suitable. Corresponding dimethicone copolyols are commercially available and are sold for example by Dow Corning under the name Dow Corning 5330 Fluid.

The teaching according to the invention naturally also comprises the fact that the dimethiconols, dimethicones and/or dimethicone copolymers can already be in the form of an emulsion. The corresponding emulsion of the dimethiconols, dimethicones and/or dimethicone copolyols can be produced both after production of the corresponding dimethiconols, dimethicones and/or dimethicone copolyols therefrom and by the conventional emulsification methods known to the person skilled in the art. To this end both cationic, anionic, non-ionic or zwitterionic surfactants and emulsifiers as auxiliary substances can be used as auxiliary agents to produce the corresponding emulsions. The emulsions of the dimethiconols, dimethicones and/or dimethicone copolyols can of course also be produced directly by means of an emulsion polymerization method. Such methods too are well known to the person skilled in the art.

If the dimethiconols, dimethicones and/or dimethicone copolyols are used as an emulsion, then according to the invention the droplet size of the emulsified particles is 0.01 to 10,000 μm, preferably 0.01 to 100 μm, more preferably 0.01 to 20 μm and particularly preferably 0.01 to 10 μm. The particle size is determined by the light scattering method.

If branched dimethiconols, dimethicones and/or dimethicone copolyols are used, this should be understood to mean that the branching is greater than a random branching that occurs by chance due to impurities in the various monomers. Within the meaning of the present invention branched dimethiconols, dimethicones and/or dimethicone copolyols are therefore understood to have a degree of branching greater than 0.01%. A degree of branching greater than 0.1% is preferred, particularly preferably greater than 0.5%. The degree of branching is determined from the ratio of unbranched monomers to branched monomers, in other words the amount of trifunctional and tetrafunctional siloxanes. Both low-branched and highly branched dimethiconols, dimethicones and/or dimethicone copolyols can be particularly preferred according to the invention.

Particularly suitable silicones are amino-functional silicones, in particular the silicones that are grouped together under the INCI name amodimethicones. It is therefore preferable according to the invention for the agents according to the invention additionally to include at least one amino-functional silicone. These are understood to be silicones having at least one, optionally substituted, amino group. Under the INCI declaration these silicones are known as amodimethicones and they are available for example in the form of an emulsion as the commercial product Dow Corning® 939 or as the commercial product Dow Corning® 949 mixed with a cationic and a non-ionic surfactant.

Amino-functional silicones are preferably used that have an amine value above 0.25 meq/g, preferably above 0.3 meq/g and in particular preferably above 0.4 meq/g. The amine value denotes the milli-equivalents of amine per gram of the amino-functional silicone. It can be determined by titration and also specified in the unit mg KOH/g.

The agents include the silicones preferably in amounts from 0.01 wt. % to 15 wt. %, more preferably from 0.05 to 2 wt. %, relative to the total agent.

As a care substance from a different class of compounds the agent can for example include at least one protein hydrolysate and/or a derivative thereof.

Protein hydrolysates are mixtures of products which are obtained by acidically, basically or enzymatically catalyzed breakdown of proteins. According to the invention the term protein hydrolysates is also understood to include total hydrolysates and individual amino acids and derivatives thereof as well as mixtures of different amino acids. The molecular weight of the protein hydrolysates for use according to the invention is between 75, the molecular weight for glycine, and 200,000; the molecular weight is preferably 75 to 50,000 and particularly preferably 75 to 20,000 daltons.

According to the invention protein hydrolysates of both plant and animal or marine or synthetic origin can be used.

Animal protein hydrolysates are for example elastin, collagen, keratin, silk and milk protein hydrolysates, which can also be present in the form of salts. Such products are sold for example under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex), Sericin (Pentapharm) and Kerasol® (Croda).

The protein hydrolysates are included in the agents according to the invention in concentrations for example from 0.01 wt. % to 20 wt. %, preferably from 0.05 wt. % to 15 wt. % and particularly preferably in amounts from 0.05 wt. % to 5 wt. %, relative in each case to the total application preparation.

The agent according to the invention can furthermore include at least one vitamin, provitamin, vitamin precursor and/or derivative thereof as a care substance.

Such vitamins, provitamins and vitamin precursors are preferred according to the invention that are conventionally assigned to groups A, B, C, E, F and H.

The group of substances classed as vitamin A includes retinol (vitamin A₁) and 3,4-didehydroretinol (vitamin A₂). β-Carotene is the retinol provitamin. Suitable vitamin A components according to the invention are for example vitamin A acid and esters thereof, vitamin A aldehyde and vitamin A alcohol and esters thereof such as the palmitate and acetate. The agents include the vitamin A component preferably in amounts from 0.05 to 1 wt. %, relative to the total application preparation.

The vitamin B group or the vitamin B complex includes inter alia vitamin B₁ (thiamine), vitamin B₂ (riboflavin), vitamin B₃ (nicotinic acid and nicotinic acid amide (niacinamide)), vitamin B₅ (pantothenic acid, panthenol and pantolactone), vitamin B₆ (pyridoxine as well as pyridoxamine and pyridoxal), vitamin C (ascorbic acid), vitamin E (tocopherols, in particular α-tocopherol), vitamin F (linoleic acid and/or linolenic acid), vitamin H.

The agents according to the invention preferably include vitamins, provitamins and vitamin precursors from groups A, B, C, E and H. Panthenol, pantolactone, pyridoxine and derivatives thereof as well as nicotinic acid amide and biotin are more preferred.

D-Panthenol, optionally in combination with at least one of the aforementioned silicone derivatives, is particularly preferably used as a care substance.

Like the addition of glycerol and/or propylene glycol, the addition of panthenol increases the flexibility of the polymer film formed on application of the agent according to the invention. If a particularly flexible hold is desired, the agents according to the invention can therefore include panthenol instead of or in addition to glycerol and/or propylene glycol. In a preferred embodiment the agents according to the invention include panthenol, preferably in an amount from 0.05 to 10 wt. %, more preferably 0.1 to 5 wt. %, relative in each case to the total agent.

The agents used according to the invention can moreover include at least one plant extract as a care substance.

These extracts are conventionally produced by extraction of the entire plant. It can also be preferable in individual cases, however, to produce the extracts exclusively from flowers and/or leaves of the plant.

The extracts from green tea, oak bark, stinging nettle, witch hazel, hops, henna, chamomile, burdock, horsetail, whitethorn, lime blossom, almond, aloe vera, pine, horse chestnut, sandalwood, juniper, coconut, mango, apricot, lemon, wheat, kiwi, melon, orange, grapefruit, sage, rosemary, birch, mallow, lady's smock, wild thyme, yarrow, thyme, melissa, restharrow, coltsfoot, marshmallow, meristem, ginseng and ginger root are preferred above all according to the invention.

It can furthermore be preferable to use mixtures of a plurality of different plant extracts, in particular two, in the agents used according to the invention.

Mono- or oligosaccharides can also be used as a care substance in the agents according to the invention.

Both monosaccharides and oligosaccharides, such as for example cane sugar, lactose and raffinose, can be used. The use of monosaccharides is preferred according to the invention. Of the monosaccharides, compounds including 5 or 6 carbon atoms are preferred in turn.

Suitable pentoses and hexoses are for example ribose, arabinose, xylose, lyxose, allose, altrose, glucose, mannose, gulose, idose, galactose, talose, fucose and fructose. Arabinose, glucose, galactose and fructose are preferably used carbohydrates. Glucose, which is suitable both in the D-(+)- or L-(−)-configuration or as a racemate, is particularly preferably used. Derivatives of these pentoses and hexoses, such as the corresponding aldonic and uronic acids (sugar acids), sugar alcohols and glycosides, can moreover also be used according to the invention. Preferred sugar acids are gluconic acid, glucuronic acid, saccharic acid, mannosaccharic acid and mucic acid. Preferred sugar alcohols are sorbitol, mannitol and dulcitol. Preferred glycosides are methyl glucosides.

As the mono- or oligosaccharides that are used are conventionally obtained from natural raw materials such as starch, they generally have the configurations corresponding to these raw materials (for example D-glucose, D-fructose and D-galactose).

The mono- or oligosaccharides are preferably included in the agents according to the invention in an amount from 0.1 to 8 wt. %, in particular preferably 1 to 5 wt. %, relative to the total application preparation.

The agent can moreover include at least one lipid as a care substance.

Lipids that are suitable according to the invention are phospholipids, for example soy lecithin, egg lecithin and cephalins, and the substances known under the INCI names Linoleamidopropyl PG-Dimonium Chloride Phosphate, Cocamidopropyl PG-Dimonium Chloride Phosphate and Stearamidopropyl PG-Dimonium Chloride Phosphate. These are sold by Mona for example under the commercial names Phospholipid EFA®, Phospholipid PTC® and Phospholipid SV®. The agents according to the invention include the lipids preferably in amounts from 0.01 to 10 wt. %, in particular 0.1 to 5 wt. %, relative to the total application preparation.

Oil bodies are also suitable as a care substance.

The natural and synthetic cosmetic oil bodies include, for example:
vegetable oils. Examples of such oils are sunflower oil, olive oil, soybean oil, rapeseed oil, almond oil, jojoba oil, orange oil, wheat germ oil, peach kernel oil and the liquid components of coconut butter. Other triglyceride oils are also suitable, however, such as the liquid components of beef fat and synthetic triglyceride oils
liquid paraffin oils, isoparaffin oils and synthetic hydrocarbons and also di-n-alkyl ethers having in total between 12 and 36 C atoms, in particular between 12 and 24 C atoms, such as for example di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether and also di-tert-butyl ether, diisopentyl ether, di-3-ethyl decyl ether, tert-butyl-n-octyl ether, isopentyl-n-octyl ether and 2-methyl pentyl-n-octyl ether. The compounds available as commercial products, 1,3-di-(2-ethylhexyl) cyclohexane (Cetiol® S) and di-n-octyl ether (Cetiol® OE), can be preferred.
ester oils. Ester oils refer to the esters of C₆-C₃₀ fatty acids with C₂-C₃₀ fatty alcohols. The monoesters of fatty acids with alcohols having 2 to 24 C atoms are preferred. More preferred according to the invention are isopropyl myristate (Rilanit® IPM), isononanoic acid C₁₆₋₁₈ alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V).
dicarboxylic acid esters such as di-n-butyl adipate, di-(2-ethylhexyl) adipate, di-(2-ethylhexyl) succinate and diisotridecyl acelate and also diol esters such as ethylene glycol dioleate, ethylene glycol diisotridecanoate, propylene glycol di-(2-ethyl hexanoate), propylene glycol diisostearate, propylene glycol dipelargonate, butanediol diisostearate, neopentyl glycol dicaprylate,
symmetrical, asymmetrical or cyclic esters of carbonic acid with fatty alcohols, as described for example in DE-OS 197 56 454, glycerol carbonate or dicaprylyl carbonate (Cetiol® CC),
tri-fatty acid esters of saturated and/or unsaturated linear and/or branched fatty acids with glycerol,
fatty acid partial glycerides, namely monoglycerides, diglycerides and technical mixtures thereof. If technical products are used, small amounts of triglycerides may also be included for production reasons. The partial glycerides preferably obey formula (D4-I),

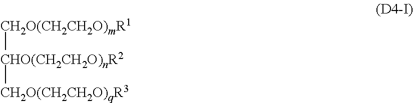

in which R¹, R² and R³ independently of one another denote hydrogen or a linear or branched, saturated and/or unsaturated acyl residue having 6 to 22, preferably 12 to 18, carbon atoms, with the proviso that at least one of these groups denotes an acyl residue and at least one of these groups denotes hydrogen. The sum (m+n+q) denotes 0 or numbers from 1 to 100, preferably 0 or 5 to 25. R¹ preferably denotes an acyl residue and R² and R³ hydrogen and the sum (m+n+q) is preferably 0. Typical examples are mono- and/or diglycerides based on hexanoic acid, octanoic acid, 2-ethylhexanoic acid, decanoic acid, lauric acid, isotridecanoic acid, myristic acid, palmitic acid, palmoleic acid, stearic acid, isostearic acid, oleic acid, elaidic acid, petroselic acid, linoleic acid, linolenic acid, elaeostearic acid, eicosanoic acid, gadoleic acid, docosanoic acid and erucic acid and technical mixtures thereof. Oleic acid monoglycerides are preferably used.

The amount of natural and synthetic cosmetic oil bodies used in the agents used according to the invention is conventionally 0.1 to 30 wt. %, relative to the total application preparation, preferably 0.1 to 20 wt. % and in particular 0.1 to 15 wt. %.

Although each of the specified care substances in itself already gives rise to a satisfactory result, all embodiments in which the agent includes a plurality of care substances, including examples from different groups, are also included in the context of the present invention.

Through the addition of a UV filter both the agents themselves and also the treated fibers can be protected from damaging influences of UV radiation. At least one UV filter is therefore preferably added to the agent. There are no general restrictions on the suitable UV filters in terms of their structure and their physical properties. In fact all UV filters that can be used in the cosmetics sector whose absorption maximum is in the UVA (315-400 nm), UVB (280-315 nm) or UVC (<280 nm) range are suitable. UV filters having an absorption maximum in the UVB range, in particular in the range from approximately 280 to approximately 300 nm, are particularly preferred.

The preferred UV filters according to the invention can be selected for example from substituted benzophenones, p-aminobenzoic acid esters, diphenyl acrylic acid esters, cinnamic acid esters, salicylic acid esters, benzimidazoles and o-aminobenzoic acid esters.

Examples of UV filters for use according to the invention are 4-aminobenzoic acid, N,N,N-trimethyl-4-(2-oxoborn-3-ylidene methyl)aniline methyl sulfate, 3,3,5-trimethyl cyclohexyl salicylate (homosalate), 2-hydroxy-4-methoxybenzophenone, 2-phenylbenzimidazole-5-sulfonic acid and potassium, sodium and triethanolamine salts thereof, 3,3′-(1,4-phenylenedimethylene)-bis(7,7-dimethyl-2-oxobicyclo-[2.2.1]hept-1-yl-methanesulfonic acid) and salts thereof, 1-(4-tert-butylphenyl)-3-(4-methoxyphenyl)propane-1,3-dione, α-(2-oxoborn-3-ylidene)toluene-4-sulfonic acid and salts thereof, ethoxylated 4-aminobenzoic acid ethyl ester (PEG-25 PABA; Uvinul® P 25), 4-dimethylaminobenzoic acid-2-ethylhexyl ester, salicylic acid-2-ethylhexyl ester, 4-methoxycinnamic acid isopentyl ester, 4-methoxycinnamic acid-2-ethylhexyl ester, 2-hydroxy-4-methoxybenzophenone-5-sulfonic acid and the sodium salt thereof (benzophenone-4; Uvinul® MS 40; Uvasorb® S 5), 3-(4′-methylbenzylidene)-D,L-camphor, 3-benzylidene camphor, 4-isopropylbenzyl salicylate, 2,4,6-trianilino-(p-carbo-2′-ethylhexyl-1′-oxi)-1,3,5-triazine, 3-imidazol-4-yl acrylic acid and ethyl esters thereof, polymers of N-{(2 and 4)-[2-oxoborn-3-ylidene methyl]benzyl}acrylamide, 2,4-dihydroxybenzophenone, 1,1′-diphenylacrylonitrilic acid-2-ethylhexyl ester, o-aminobenzoic acid menthyl ester, 2,2′,4,4′-tetrahydroxybenzophenone, 2,2′-dihydroxy-4,4′-dimethoxybenzophenone, 2,2′-dihydroxy-4,4′-dimethoxybenzophenone-5-sodium sulfonate and 2-cyano-3,3-diphenylacrylic acid-2′-ethylhexyl ester. 2-Hydroxy-4-methoxybenzophenone-5-sulfonic acid and the sodium salt thereof and/or ethoxylated 4-aminobenzoic acid ethyl ester are preferred.

The UV filters are conventionally included in amounts from 0.01 to 5 wt. %, relative to the total application preparation. Amounts from 0.1 to 2.5 wt. % are preferred.

In a particular embodiment the agent used according to the invention furthermore includes one or more substantive dyes. This allows the treated keratinic fibers not only to be temporarily structured but at the same time also to be colored through the use of the agent. This can be desirable in particular if only a temporary coloration is desired, for example with striking fashion colors, which can be removed again from the keratinic fibers simply by washing.

Substantive dyes are conventionally nitrophenylene diamines, nitroaminophenols, azo dyes, anthraquinones or indophenols. Preferred substantive dyes are the compounds known under the international names or trade names HC Yellow 2, HC Yellow 4, HC Yellow 5, HC Yellow 6, HC Yellow 12, Acid Yellow 1, Acid Yellow 10, Acid Yellow 23, Acid Yellow 36, HC Orange 1, Disperse Orange 3, Acid Orange 7, HC Red 1, HC Red 3, HC Red 10, HC Red 11, HC Red 13, Acid Red 33, Acid Red 52, HC Red BN, Pigment Red 57:1, HC Blue 2, HC Blue 11, HC Blue 12, Disperse Blue 3, Acid Blue 7, Acid Green 50, HC Violet 1, Disperse Violet 1, Disperse Violet 4, Acid Violet 43, Disperse Black 9, Acid Black 1 and Acid Black 52 as well as 1,4-diamino-2-nitrobenzene, 2-amino-4-nitrophenol, 1,4-bis-(B-hydroxyethyl)-amino-2-nitrobenzene, 3-nitro-4-(β-hydroxyethyl)aminophenol, 2-(2′-hydroxyethyl)amino-4,6-dinitrophenol, 1-(2′-hydroxyethyl)amino-4-methyl-2-nitrobenzene, 1-amino-4-(2′-hydroxyethyl)amino-5-chloro-2-nitrobenzene, 4-amino-3-nitrophenol, 1-(2′-ureidoethyl)amino-4-nitrobenzene, 4-amino-2-nitro-diphenylamine-2′-carboxylic acid, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid and 2-chloro-6-ethylamino-1-hydroxy-4-nitrobenzene. Cationic substantive dyes are preferably used. Of those, the following are more preferred:
(a) cationic triphenylmethane dyes, such as for example Basic Blue 7, Basic Blue 26, Basic Violet 2 and Basic Violet 14,
(b) aromatic systems substituted with a quaternary nitrogen group, such as for example Basic Yellow 57, Basic Red 76, Basic Blue 99, Basic Brown 16 and Basic Brown 17, and
(c) substantive dyes including a heterocyclic compound having at least one quaternary nitrogen atom, such as for example those mentioned in EP-A2-998 908, to which reference is explicitly made here, in claims 6 to 11.

The dyes that are also known under the names Basic Yellow 87, Basic Orange 31 and Basic Red 51 are particularly preferred cationic substantive dyes of group (c). The cationic substantive dyes which are sold under the trademark Arianor® are likewise particularly preferred cationic substantive dyes according to the invention.

In this embodiment the agents used according to the invention preferably include the substantive dyes in an amount from 0.001 to 20 wt. %, relative to the total agent.

It is preferable according to the invention for the agents used according to the invention to be free from oxidation dye precursors. Oxidation dye precursors are divided into developer components and coupler components. The developer components form the actual dyes with one another under the influence of oxidizing agents or of atmospheric oxygen or by coupling with one or more coupler components.

The agents used according to the invention can be formulated in all conventional forms for styling agents, for example in the form of solutions, which can be applied to the hair as a hair lotion or pump or aerosol spray, in the form of creams, emulsions, waxes, gels or surfactant-including foaming solutions or other preparations that are suitable for application on the hair.

Hair creams and hair gels generally include structuring agents and/or thickening polymers, which serve to impart the desired consistency to the products. Structuring agents and/or thickening polymers are typically used in an amount from 0.1 to 10 wt. %, relative to the total product. Amounts from 0.5 to 5 wt. %, in particular 0.5 to 3 wt. %, are preferred.

The agents used according to the invention are preferably presented as a pump spray, aerosol spray, pump foam or aerosol foam.

To this end the agents according to the invention are presented in a dispensing device, which is either a compressed gas cylinder (aerosol container) additionally filled with a propellant or a non-aerosol container.

Compressed gas cylinders, with the aid of which a product is distributed through a valve via the internal gas pressure in the cylinder, are by definition termed aerosol containers. In contrast to the aerosol definition, a “non-aerosol container” is defined as a container under normal pressure, with the aid of which a product is distributed through a pump system by means of a mechanical action.

The agents used according to the invention are in particular preferably presented as an aerosol hair foam or aerosol hair spray. The agent according to the invention therefore preferably additionally includes at least one propellant.

Propellants that are suitable according to the invention are selected for example from N₂O, dimethyl ether, CO₂, air, alkanes having 3 to 5 carbon atoms, such as propane, n-butane, isobutane, n-pentane and isopentane, and mixtures thereof. Dimethyl ether, propane, n-butane, isobutane and mixtures thereof are preferred.

According to a preferred embodiment the specified alkanes, mixtures of the specified alkanes or mixtures of the specified alkanes with dimethyl ether are used as the sole propellant. However the invention expressly also comprises the incorporation of propellants of the chlorofluorocarbon type, but in particular fluorocarbons.

In the given spray device the sizes of the aerosol droplets or foam bubbles and the size distribution in each case can be adjusted by the ratio of propellant to the other constituents of the preparations.

The amount of propellant used varies according to the specific composition of the agent, the packaging used and the desired product type, for instance hair spray or hair foam. If conventional spray devices are used, aerosol foam products preferably include the propellant in amounts from 1 to 35 wt. %, relative to the total product. Amounts from 2 to 30 wt. %, in particular from 3 to 15 wt. %, are more preferred. Aerosol sprays generally include larger amounts of propellant. In this case the propellant is preferably used in an amount from 30 to 98 wt. %, relative to the total product. Amounts from 40 to 95 wt. %, in particular from 50 to 95 wt. %, are more preferred.

The aerosol products can be produced in the conventional manner. All constituents of the individual agent with the exception of the propellant are generally introduced into a suitable pressure-resistant container. This is then closed with a valve. Finally the desired amount of propellant is added using conventional techniques.

Isopentane is preferably suitable as a propellant for expanding agents in gel form in a two-chamber aerosol container, it being incorporated into the agents according to the invention and introduced into the first chamber of the two-chamber aerosol container. At least one further propellant that is different from isopentane and that establishes a higher pressure in the two-chamber aerosol container than isopentane is introduced into the second chamber of the two-chamber aerosol container. The propellants of the second chamber are preferably selected from N₂O, dimethyl ether, CO₂, air, alkanes having 3 or 4 carbon atoms (such as propane, n-butane, isobutane) and mixtures thereof.

Aerosol hair foams or aerosol hair sprays including the agents according to the invention as described above and at least one propellant are a preferred embodiment of the agents used according to the invention.

Preferred agents used according to the invention and propellants of the aerosol hair foam or aerosol hair spray and the amounts of propellant in each case correspond to the details specified above.

The use according to the invention of the agents described above makes it possible to improve the color retention of oxidatively dyed keratinic fibers.

The present application also provides a method for treating keratin-including fibers, in particular human hair, comprising the following steps:

i) performing an oxidative dyeing of the keratin-including fibers,
ii) applying an agent including, in a cosmetically acceptable carrier,
(a) at least one non-ionic, propylene oxide-modified starch
   and
(b) at least one film-forming anionic and/or fixing anionic polymer comprising at least one structural unit of formula (I) and at least one structural unit of formula (II),

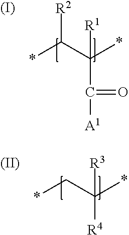

in which
R¹ and R² independently of one another denote a hydrogen atom or a methyl group, with the proviso that R¹ and R² do not simultaneously denote a methyl group,
R³ denotes a hydrogen atom or a methyl group,
R⁴ denotes a carbamoyl group, a linear or branched (C₄ to C₁₂) alkyl aminocarbonyl group, a linear or branched (C₄ to C₁₂) alkyl aminoethyl aminocarbonyl group, a linear or branched (C₄ to C₁₂) alkyl aminopropyl aminocarbonyl group, a linear or branched (C₄ to C₁₂) alkyloxycarbonyl group, a linear or branched (C₄ to C₁₂) alkyl aminoethyl oxycarbonyl group, a linear or branched (C₄ to C₁₂) alkyl aminopropyl oxycarbonyl group, a linear or branched (C₂ to C₁₂) acyloxy group,
A¹ denotes a hydroxyl group or an organic residue having at least one sulfonic acid group, which binds to the structural fragment via an oxygen atom or an NH group, to the oxidatively dyed keratin-including fibers.

The aforementioned dispensing devices (see above) are preferred according to the invention.

In a preferred embodiment of the method according to the invention the agent is applied in step ii) to the keratin-including fibers as a spray. It is preferable according to the invention for the keratin-including fibers to be shaped and for this shape to be fixed by the agent applied in step ii).

The examples below are intended to illustrate the subject matter of the present invention without in any way limiting it.

### EXAMPLES

Unless otherwise specified, the quantities given below are percentages by weight.

The following formulations were prepared by mixing the specified raw materials:

| Raw materials | A | B |
|---|---|---|
| PGE¹ | 10.0 | — |
| Polyvinylpyrrolidone (20%) | — | 22.5 |
| PVP/VA copolymer 60/40 | 7.0 | 7.0 |
| Gafquat 755N² | 5.2 | 5.2 |
| Dehyquart A³ | 1.0 | 1.0 |
| Lactic acid (80%) | 0.08 | 0.08 |
| PEG-40 hydrogenated castor oil | 0.4 | 0.4 |
| Water | to 100 | |

¹Non-ionic, propylene oxide-modified potato starch (43 wt. % active substance in water; propylene oxide content: 10 wt. %; viscosity: 64,000 mPas; weight average: 900 kDa)
²Copolymer of vinylpyrrolidone and dimethylaminoethyl methacrylate (20 wt. % active substance in water, INCI name: Polyquaternium 11)
³Trimethyl hexadecyl ammonium chloride (25 wt. % active substance in water; INCI name: Cetrimonium Chloride)

After being applied to oxidatively dyed keratinic fibers, formulation A brought about an improved color retention in comparison to formulation B.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A method of improving the color retention of oxidatively dyed keratin-including fibers keratin-including fibers, in particular human hair, comprising:
i) performing an oxidative dyeing of the keratin-including fibers,
ii) applying an agent including, in a cosmetically acceptable carrier,
(a) at least one non-ionic, propylene oxide-modified starch, and
(b) at least one film-forming and/or fixing polymer, to the oxidatively dyed keratin-including fibers.

2. The method according to claim 1, wherein the film-forming and/or fixing polymer comprises at least one structural unit of formula (I) and at least one structural unit of formula (II), 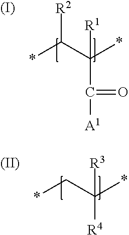
in which
R¹ and R² independently of one another denote a hydrogen atom or a methyl group, with the proviso that R¹ and R² do not simultaneously denote a methyl group,
R³ denotes a hydrogen atom or a methyl group,
R⁴ denotes a carbamoyl group, a linear or branched (C₄ to C₁₂) alkyl aminocarbonyl group, a linear or branched (C₄ to C₁₂) alkyl aminoethyl aminocarbonyl group, a linear or branched (C₄ to C₁₂) alkyl aminopropyl aminocarbonyl group, a linear or branched (C₄ to C₁₂) alkyloxycarbonyl group, a linear or branched (C₄ to C₁₂) alkyl aminoethyl oxycarbonyl group, a linear or branched (C₄ to C₁₂) alkyl aminopropyl oxycarbonyl group, a linear or branched (C₂ to C₁₂) acyloxy group,
A¹ denotes a hydroxyl group or an organic residue having at least one sulfonic acid group, which binds to the structural fragment via an oxygen atom or an NH group.

3. The method according to claim 1, wherein the non-ionic, propylene oxide-modified starch is one or more starches selected from the group consisting of a non-ionic, propylene oxide-modified tapioca starch and a non-ionic, propylene oxide-modified potato starch.

4. The method according to one claim 1, wherein the agent includes the non-ionic, propylene oxide-modified starch in an amount from 1.0 wt. % to 20 wt. % relative to the weight of the agent.

5. The method according to claim 1, wherein the non-ionic, propylene oxide-modified starch has an average molecular weight (weight-average) from 50 to 2500 kDa.

6. The method according to claim 1, wherein the non-ionic, propylene oxide-modified starch has a propylene oxide content from 1 to 20 wt. % relative to the weight of the modified starch.

7. The method according to claim 1, wherein at least one uncrosslinked, non-ionic, propylene oxide-modified starch is included as the non-ionic, propylene oxide-modified starch.

8. The method according to claim 2, wherein the film-forming and/or fixing polymer (b) includes at least one structural unit of formula (I), which is selected from the group consisting of formulae (I-1) to (I-5)

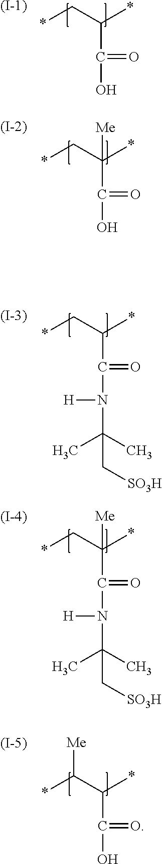

9. The method according to claim 2, wherein the film-forming and/or fixing polymer (b) includes at least one structural unit of formula (II), which is selected from the group consisting of formulae (II-1) to (II-15). 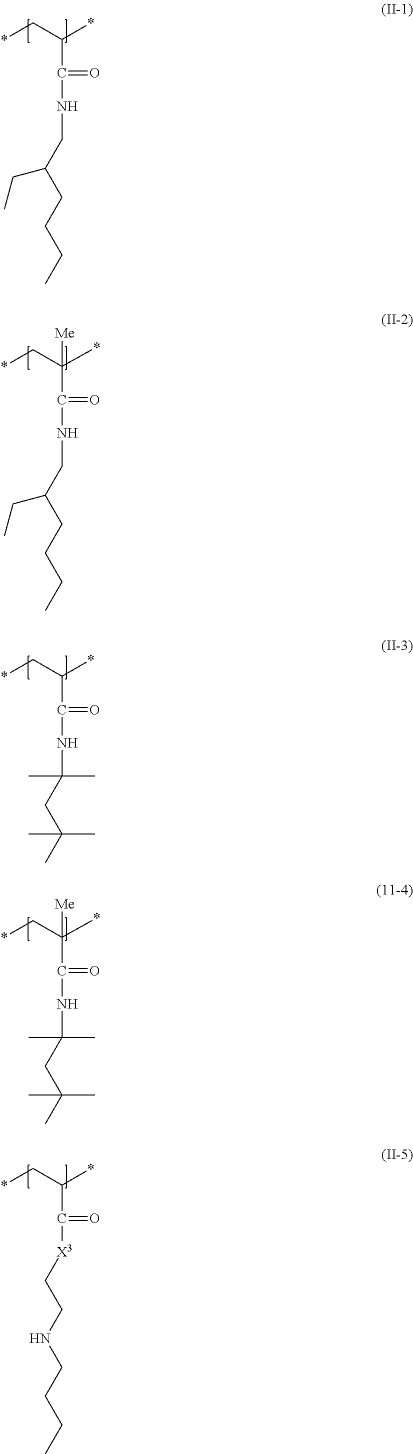 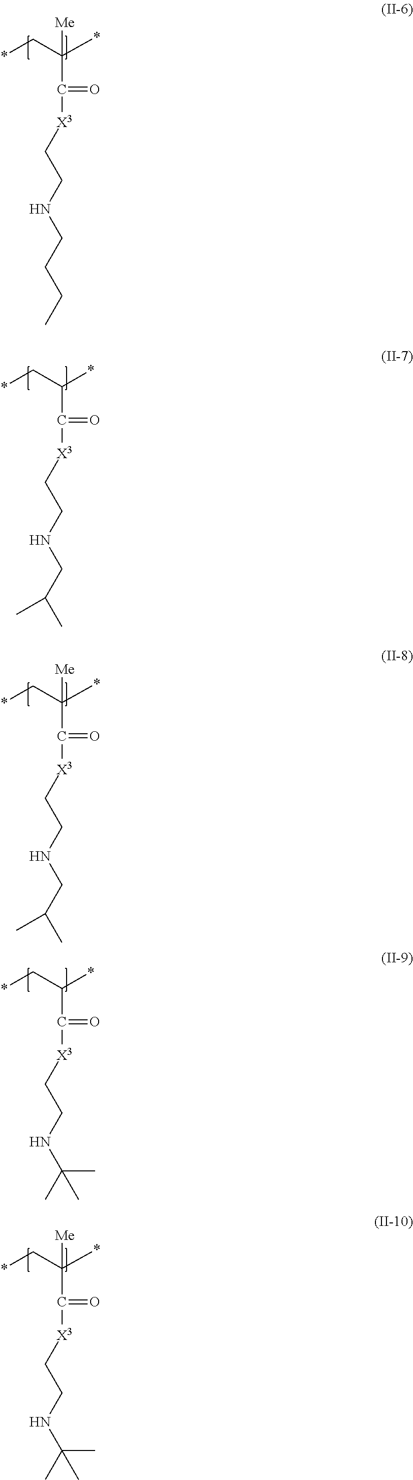 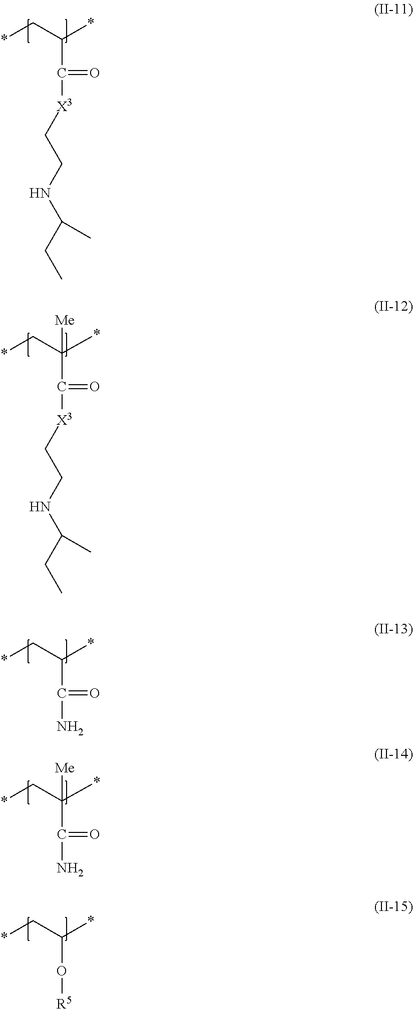
in which
X³ denotes an oxygen atom or an NH group,
R⁵ denotes a (C₂ to C₁₂) acyl group (in particular acetyl or neodecanoyl).

10. The method according to claim 1, wherein the film-forming and/or fixing polymer (b) is selected from at least one polymer of the group formed from
copolymers of acrylic acid, (C₁ to C₄) alkyl acrylates, C₄ alkyl aminoethyl methacrylate and C₈ alkyl acrylamide,
copolymers of 2-acrylamido-2-methylpropane sulfonic acid and acrylamide,
copolymers of 2-acrylamido-2-methylpropane sulfonic acid, acrylamide and acrylic acid,
copolymers of vinyl acetate and crotonic acid,
copolymers of vinyl propionate and crotonic acid,
copolymers of vinyl neodecanoate, vinyl acetate and crotonic acid,
copolymers of methacrylic acid and ethyl acrylate and tert-butyl acrylate.

11. The method according to claim 1, wherein the agent includes the film-forming and/or fixing polymers (b) in an amount from 0.5 wt. % to 10 wt. % relative to the weight of the agent.

12. The method according to claim 1, wherein the agent is in the form of an aerosol foam or an aerosol spray.

13. The method according to claim 1, wherein the agent has a pH (25° C.) from 4.0 to 9.0.
